(19) United States
(12) Patent Application Publication
(10) Pub. No.: US 2009/0324520 A1
(43) Pub. Date: Dec. 31, 2009

(54) PERSONAL-CARE ARTICLE FOR SEQUENTIALLY DISPENSING COMPOSITIONS WITH VARIABLE CONCENTRATIONS OF PARTITIONED BENEFIT OR SUSPENDED BENEFIT AGENTS

(76) Inventors: Jonathan Robert Cetti, Mason, OH (US); Christopher Dean Putman, West Chester, OH (US); Karen Lehnhoff, Cincinnati, OH (US); Richard Lee Valentino, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/361,467

(22) Filed: Jan. 28, 2009

Related U.S. Application Data
(63) Continuation-in-part of application No. 11/881,536, filed on Jul. 27, 2007.

(51) Int. Cl. A61K 8/00; A61Q 19/02; A61Q 17/04; A61Q 19/04; A61P 17/10; B65D 25/40

(52) U.S. Cl. 424/59; 424/62; 222/575

(57) ABSTRACT

The present invention relates to a personal-care article comprising a package and a personal-care product. The package comprises at least one chamber, a dispensing orifice, a first zone proximate to the dispensing orifice, and a second zone distal to the dispensing orifice. The first zone and second zone are both located in at least one chamber. The personal-care product comprises a first personal-care composition substantially disposed within the first zone and a second personal-care composition substantially disposed within the second zone. The first composition comprises a first concentration of a partitioned benefit agent or a suspended benefit agent. The second composition comprises a benefit phase comprising a second concentration of a partitioned benefit agent or a suspended benefit agent. The first concentration is different from the second concentration.

### CROSS REFERENCE TO RELATED APPLICATION

This application is a continuation-in-part of U.S. application Ser. No. 11/881,536, filed Jul. 27, 2007.

### FIELD OF THE INVENTION

The present invention relates to a personal-care article that provides a personal-care product that comprises at least two compositions each having a concentration of a partitioned benefit agent or suspended benefit agent which is noticeably distinct from the other.

### BACKGROUND OF THE INVENTION

Personal-care compositions are well known and widely used for cleansing and moisturizing skin and hair, delivering actives, hiding imperfections, to reducing the oiliness/shine, as well as, providing scent to the shower and/or the skin. The efficacy of these types of compositions is directly related to their frequency of use and level of active ingredients. In some cases, a high level of benefit agent in a personal-care composition will maintain a benefit to a consumer for several days after a single application. In this case, a full bottle of the composition with a high level of benefit agent is not needed because the continued application of personal-care composition with high level of benefit agent would not provide additional benefit to the consumer over one or two single applications. Numerous cosmetic applications require that the corresponding compositions be used at variable dose of active ingredients in the course of time. Up until now, it order to carry out these treatments, the available resources have consisted either of successive applications of decreasing active ingredient percentages in separate containers or multiplying the applications of compositions with active ingredients percentages in order to obtain the correct does for the necessary treatment. If a treatment regime contains too many steps or too many containers, consumers often habituate or tire of the regime of personal-care compositions over time. When this habituation occurs consumers often decrease or even or stop use of one personal-care product despite the benefits gained by the compliant use of the regime of personal-care products over time. With the space in the shower or bath being limited, a typical shower or bath does not have enough space, to place multiple containers of personal-care compositions so that a consumer can easily switch the use of one personal-care composition to another personal-care composition with a different level or type of benefit agent.

### SUMMARY OF THE INVENTION

The present invention relates to a personal-care article for providing at least two personal-care compositions. The personal-care article comprises a package and a personal-care product. The package comprises at least one chamber, a dispensing orifice, a first zone proximate to the dispensing orifice and a second zone distal to the dispensing orifice. The first second and the second zone are in physical contact with each other in at least one chamber. The personal-care product comprises a first personal-care composition substantially disposed within the first zone and the second personal-care composition substantially disposed within the second zone. The first personal-care composition comprises a first concentration of partitioned benefit component. The second personal-care composition comprises a second concentration of partitioned benefit component. The partitioned benefit component is selected from the group consisting of fragrances, moisturizing agents, lather producers, lather supressors, vitamins, vitamin derivatives, sunscreens, anti-wrinkle, skin soothing agents, skin lightening agents, skin darkening agents, anti-acne medicaments, essential oils, sensates, colorants and mixtures thereof. The first concentration of partitioned benefit component is different from the second concentration of partitioned benefit component.

The present invention also relates to a personal-care article for providing at least two personal-care compositions. The personal-care article comprises a package and a personal-care product. The package comprises least one chamber, a dispensing orifice, a first zone proximate to the dispensing orifice and a second zone distal to the dispensing orifice, wherein both zones are located in at least one chamber. The personal-care product comprises a first personal-care composition substantially disposed within the first zone and the second personal-care composition substantially disposed within the second zone. The first personal-care composition comprises a first concentration of a suspended benefit agent and the second personal-care composition comprises a second concentration of suspended benefit agent. The suspended benefit agents are selected from the group consisting of comprise hydrophobic benefit materials, polymers, moisturizing agents, pigments, interference pigments, pearlescent agents, particles, exfoliating particles, shiny particles, beads, hydrophobically modified non-platelet particles, microcapsules, and mixtures thereof. The first concentration of suspended benefit agent is different from the second concentration of suspended benefit agent.

Thus, the personal-care articles of the present invention comprises a personal-care product that changes in level or type of benefit material as it is dispensed from the package which overcomes the problem of a regime that involves too many steps or too many containers.

### BRIEF DESCRIPTION OF THE DRAWINGS

FIGS. 1A and 1B illustrate a personal-care article with three zones having horizontal interfaces between the compositions and the zones.

FIGS. 2A and 2B illustrate a personal-care article with two zones having diagonal interfaces between the compositions and the zones

FIGS. 3A and 3B illustrate a personal-care article with two zones having horizontal interfaces between the compositions and the zones.

### DETAILED DESCRIPTION OF THE INVENTION

The term “ambient conditions” as used herein, refers to surrounding conditions at one (1) atmosphere of pressure, 50% relative humidity, and 25° C.

As used herein, “comprising” means that other steps and other ingredients which do not affect the end result can be added. This term encompasses the terms “consisting of” and “consisting essentially of.” The compositions and methods/processes of the present invention can comprise, consist of, or consist essentially of the essential elements and limitations of the invention described herein, as well as any of the additional or optional ingredients, components, steps, or limitations described herein useful in personal-care compositions intended for topical application to the hair or skin.

The term “personal-care product,” as used herein, may include, but is not limited to: antiperspirants, deodorants, lotions (e.g. hand lotion and body lotion), skin-care compositions (e.g., face and neck lotions, serums, sprays), sunless tanners, cosmetic compositions (e.g., foundation, concealer, blush, lipstick, lip gloss), depilatories, shampoos, conditioning shampoos, hair conditioners, body washes, moisturizing body washes, shower gels, skin cleansers, cleansing milks, hair and body washes, in-shower body moisturizers, pet shampoos, shaving preparations, after-shaves, razor moisturizing/lubricating strips, razor shave-gel bars, bar soaps, cleansing compositions, feminine-care products, oral-care products, and baby-care products.

The term “personal-care composition,” as used herein, refers to compositions intended for topical application to the skin or hair. Compositions of the present invention may be leave-on formulations-in which the product is applied topically to the skin or hair and left on for a period of time, or rinse-off formulations-in which the product is applied topically to the skin or hair and then is subsequently rinsed within minutes from the skin or hair with water, or otherwise wiped off using a substrate with deposition of a portion of the composition. The personal-care composition of the present invention is typically extrudable or dispensible from a package. In other embodiments, the personal-care article may not comprise a package at all- for instance, in the case of bar soap. The personal-care compositions of the present invention can be in the form of solid, semi-solid, liquid, semi-liquid, cream, lotion or gel compositions intended for topical application to skin.

The term “package” includes any suitable container for personal-care compositions, including but not limited to a canister, bottle, tottle, tube, jar, non-aerosol pump and mixtures thereof. As used herein “tottle” refers to a bottle which rests on the neck or mouth which its contents are filled in and dispensed from, but it is also the end upon which the bottle is intended to rest or sit upon for storage by the consumer and/or for display on the store shelf, as described in the commonly owned U.S. patent application Ser. No. 11/067,443 filed on Feb. 25, 2005 to McCall, et al, entitled “Multi-phase Personal-Care Compositions, Process for Making and Providing, and Article of Commerce.”

The term “dispensing orifice,” as used herein, refers to any opening in a package through which product may be dispensed and/or applied. For example, an antiperspirant package may comprise a dispensing orifice in the form of an application surface. The application surface may be an antiperspirant product itself or the application surface may be a perforated or mesh-like dome through which antiperspirant product passes.

The term “partitioned benefit component,” or “partitioned component” as used herein refers to small molecules that has a molecular weight less than 1000 and is capable of being maintained or dispersed in a surfactant containing phase and is capable of being partitioned into two or more separate compositions. Examples of partitioned benefit components or partitioned components include but are not limited those selected from the group consisting of hydrophobic benefit material, thickening agents, fragrances, moisturizing agents, lather producers, lather suppressors, vitamins, vitamin derivatives, sunscreens, anti-wrinkle, skin soothing agents, skin lightening agents, skin tanning agents, anti-acne medicaments, essential oils, sensates (e.g. menthol), feel agents, colorants, and mixtures thereof. The term “stable” as applied to partitioned benefit components, as used herein, means that the compositions of the personal-care product that maintain at least two “separate” compositions when sitting in physical contact at ambient conditions for a period of at least 1 week according to the dialysis method described hereinafter. By “separate,” it is meant that there is substantially no mixing of the benefit agents of two compositions proximate to each other with the personal-care article, such that less than 30% of the concentration of a partitioned benefit agent of interest within the first composition migrates to the second composition proximate to first composition. The partitioned components of interest are detected by the Gas Chromatograph method described hereinafter. For example that is not considered “stable” as defined is the partitioned component Triethyl Citrate, which has a ClogP of 1.49. Using the dialysis method, analytical measurements indicate that 42.6% of the Triethyl Citrate concentration had migrated from a composition containing Triethyl Citrate into the opposite side of the dialysis cell, a composition not containing Triethyl Citrate. A further example that is not considered “stable” as defined is the partitioned component Glycerine, which has a ClogP of −2.32. Using the dialysis method, analytical measurements indicate that 60% of the Glycerine concentration had migrated from a composition containing Glycerine into the opposite side of the dialysis cell, a composition not containing Glycerine.

The term “structured,” as used herein means having a rheology that confers stability on the personal-care composition. The degree of structure is determined by characteristics determined by one or more of the following methods the Yield Stress Method, or the Zero Shear Viscosity Method or by the Ultracentrifugation Method, all in the Test Methods below. Accordingly, a surfactant phase of the composition of the present invention is considered “structured,” if the surfactant phase has one or more of the following properties described below according to Yield Stress Method, or the Zero Shear Viscosity Method or by the Ultracentrifugation Method. A surfactant phase is considered to be structured, if the phase has one or more of the following characteristics:
A. a Yield Stress of greater than about 0.1 Pascal (Pa), more preferably greater than about 0.5 Pa, even more preferably greater than about 1.0 Pa, still more preferably greater than about 2.0 Pa, still even more preferably greater than about 3 Pa, and even still even more preferably greater than about 5 Pa as measured by the Yield Stress and Zero Shear Viscosity Method described hereafter:
B. a Zero Shear Viscosity of at least about 500 Pascal-seconds (Pa-s), preferably at least about 1,000 Pa-s, more preferably at least about 1,500 Pa-s, even more preferably at least about 2,000 Pa-s; or
C. a Structured Domain Volume Ratio as measured by the Ultracentrifugation Method described hereafter, of greater than about 40%, preferably at least about 45%, more preferably at least about 50%, more preferably at least about 55%, more preferably at least about 60%, more preferably at least about 65%, more preferably at least about 70%, more preferably at least about 75%, more preferably at least about 80%, even more preferably at least about 85%.

The term “surfactant component” as used herein means the total of all anionic, nonionic, amphoteric, zwitterionic and cationic surfactants in a phase. When calculations are based on the surfactant component, water and electrolyte are excluded from the calculations involving the surfactant component, since surfactants as manufactured typically are diluted and neutralized.

“Suspended benefit agent” as used herein are larger molecules having a molecular weight larger than 1000 or are “particulates” or “particles.” Examples of suspended benefit agents include but are not limited to hydrophobic benefit materials, polymers, moisturizing agents, pigments, interference pigments, pearlescent agents, particles, exfoliating particles, shiny particles, beads, hydrophobically modified non-platlet particles, microcapsules, and mixtures thereof. The term “stable” as it applies to suspended benefit agents, as used herein, means that the compositions of the personal-care product maintain at least two separate compositions when sitting in physical contact at 120° F. (48.9° C.) for a period of at least 10 days. By “separate,” it is meant that there is substantially no mixing of the benefit agents of the two compositions proximate to each other with the personal-care article, such that less than 25% of the concentration of the larger molecules having a molecular weight larger than 1000 or particles of interest within the first composition migrates to the second composition proximate to the first composition.

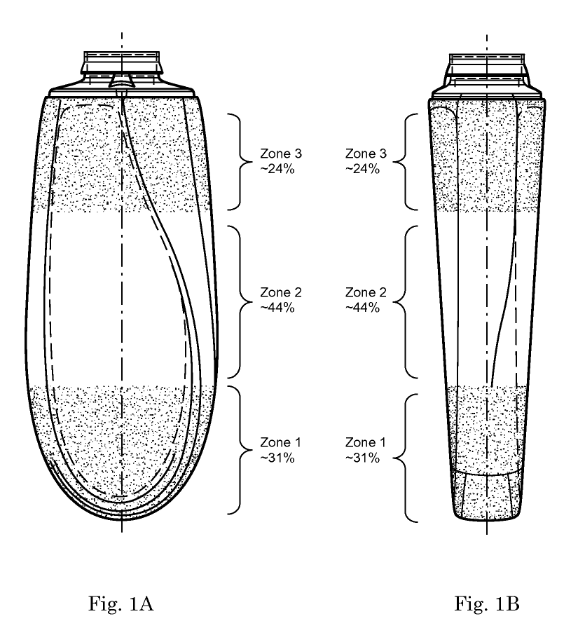

As used herein the term “zone” is a boundary within a package which corresponds to a composition of the personal-care product. A zone within in package is in direct physical contact with another zone within a package, such that the compositions corresponding to the zone are in direct physical contact with one another. The interface between the zones can be distinct or gradual. The zone can be defined by a percentage of the package volume and a zone comprises at least 10% of the package volume of a given package, excluding the volume of the package corresponding to the closure, as shown in FIGS. 1A and 1B, FIGS. 2A and 2B and FIGS. 3A and 3B of the present invention.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about.”

All molecular weights as used herein are weight average molecular weights expressed as grams/mole, unless otherwise specified.

The personal-care product comprises at least two personal-care compositions, each composition having noticeably distinct benefit material concentrations. These distinct concentrations can be dispensed sequentially from the package. For example, a package may dispense a composition with a high level of benefit material comprising exfoliating beads, followed by a composition with a medium level of skin tanning benefit material, followed by a composition with a lower level of hydrophobic benefit material. Thus, the personal-care product changes in benefit as it is dispensed from the package which overcomes the problem of a regime that involves too many steps or too many containers.

It is known in the art that multiple compositions can be held separate such as is disclosed in U.S. Pat. No. 6,787,511 to Patel (hereinafter referred to as the '511 patent), for example, which two aqueous compositions are contained within a single-chamber package, wherein, when standing, the aqueous compositions form two or more visibly distinct aqueous compositions and, when agitated, the composition forms a visible single-composition product.

In contrast to the present invention, the product described in the '511 patent is intended to be shaken to deliver the intended benefit. The viscosities of the individual compositions are disclosed in the '511 patent are such that the viscosity of the mixture is greater than the viscosity of either of the layers alone. The viscosities of the two compositions of the '511 patent art are represented by LYNX® Speed Shower Shake (containing maltodextrin, sodium chloride, surfactant, water and minors). The viscosities of the two compositions by LYNX® Speed Shower Shake were measured and found to be 26 centipoise for the lower composition and 1,203 centipoise for the upper composition, which are significantly lower than the disclosed viscosities of the compositions described in the subject invention. Thus, agitation of the product described in the '511 patent is needed to deliver the viscosity appropriate for the intended use.

The present invention relates to a personal-care article for providing at least two personal-care compositions. The personal-care article comprises a package and a personal-care product. The package comprises at least one chamber, a dispensing orifice, a first zone proximate to the dispensing orifice and a second zone distal to the dispensing orifice; both zones are located in at least one chamber. The personal-care product comprises a first personal-care composition substantially disposed within the first zone and the second personal-care composition substantially disposed within the second zone. In one aspect, the first zone is in physical contact with the second zone within the package. In one aspect, the first personal-care composition is in physical contact with the second personal-care composition within the package. In one aspect, the personal-care article is not intended to be shaken such that the first personal-care composition mixes with the second personal-care composition prior to dispensing the personal-care compositions within the package.

The personal-care article for dispensing and or applying at least two personal-care compositions comprises a package that comprises at least two zones with at least two personal-care compositions substantially disposed within the respective zones in at least one chamber. The number of zones with a package and thus, the number of personal-care compositions disposed within the respective zone can vary in number. For example, the package may have three zones and three personal-care composition within the respective zones; four zones and four compositions, five zones and five compositions, and so on. In one aspect, the personal-care article comprises a third zone medial to the dispensing orifice. In one aspect, the personal-care article comprising a third personal-care composition substantially disposed within the third zone; the third personal-care composition comprising a benefit phase comprising third concentration of the partitioned benefit agent or suspended benefit agent is different from the first concentration of the partitioned benefit agent or suspended benefit agent and the second concentration of the partitioned benefit agent or suspended benefit agent. In another aspect, the first zone, the second zone and the third zone comprise an equal percentage, by volume, of the package.

In another aspect, each personal-care composition may comprise a dye, colorant or the like, such that each personal-care composition is a distinct color or hue. For example, the first personal-care composition can be a yellow color, the second personal-care composition can be a orange color and the third personal-care composition can be a purple color.

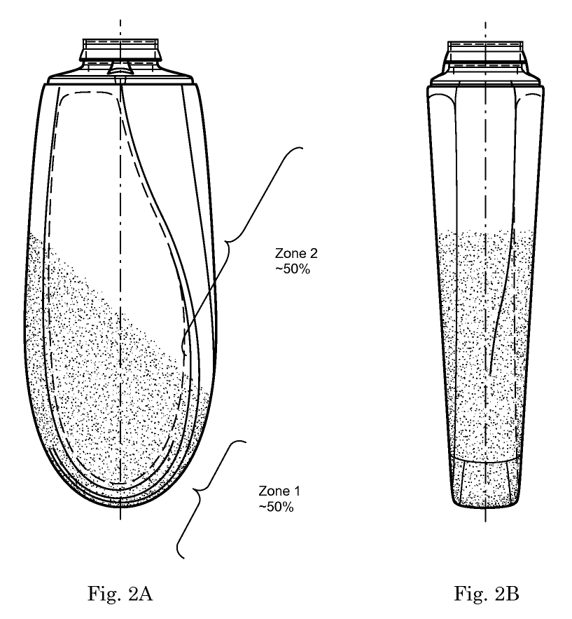

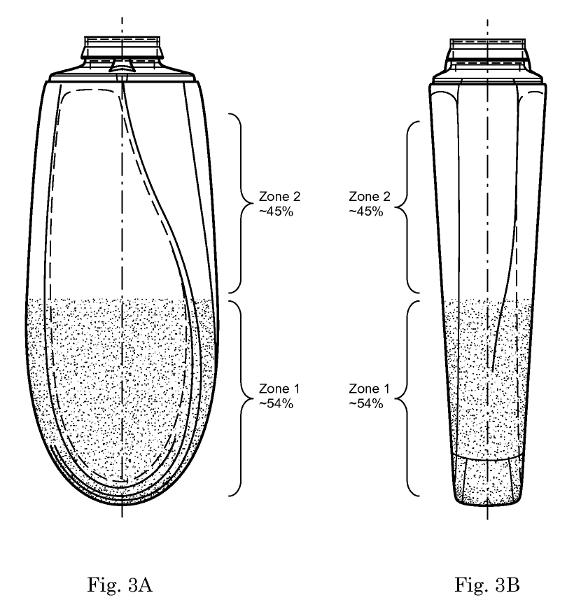

FIGS. 1A and 1B illustrate a personal-care article with three zones with horizontal interfaces between the zones. As shown in FIGS. 1A and 1B, zone 1 is approximately 31% of the package volume, zone 2 is approximately 44% of the package volume and zone 3 is approximately 24% of the package volume. FIGS. 2A and 2B illustrate a personal-care article with two zones having diagonal interfaces between the compositions and the zones. As shown in FIGS. 2A and 2B, the length of zone 1 and 2 are approximately 50% of the package volume. FIGS. 3A and 3B illustrate a personal-care article with two zones having horizontal interfaces between the compositions and the zones. As shown in FIGS. 3A and 3B, zone 1 is 54% of the package volume and zone 2 is approximately 45% of the package volume.

The first personal-care composition comprises a first concentration of a partitioned benefit agent and the second personal-care composition comprises a second concentration of partitioned benefit agent. The partitioned benefit agent is selected from the group consisting of hydrophobic benefit material, thickening agents, fragrances, moisturizing agents, lather producers, vitamins, vitamin derivatives, sunscreens, anti-wrinkle, skin soothing agents, skin lightening agents, skin tanning agents, anti-acne medicaments, essential oils, sensates, colorants and mixtures thereof. The first concentration of partitioned benefit agent is different from the second concentration of partitioned benefit agent. In another embodiment, the first partitioned benefit agent in the first personal-care composition is different from the second partitioned benefit agent in the second personal-care composition. In another embodiment, the second personal-care composition could also comprise a second partitioned benefit agent that is different from the partitioned benefit agent comprised in the first composition.

The personal-care compositions of the present invention comprise partitioned benefit agents. The Inventors believe that stability of a personal-care composition can be enhanced if one chooses to use partitioned benefit agents in personal-care composition that have a higher ClogP and are more hydrophobic and to avoid partitioned components that have a lower ClogP and are more hydrophilic. Preferably, the ClogP of the partitioned benefit agent is at least 2.

Furthermore, the inventors believe that the stability of a personal-care composition can be further enhanced if one chooses to use partitioned benefit agents in personal-care compositions that have a smaller molar volume and are more stable when dispersed or maintained in the surfactant phase and to avoid partitioned components that have a higher molar volume and are less stable when dispersed or maintained in the surfactant phase The molar volume as determined hereinafter is at least from about 50, or at least from about 75, or at least from about 100 cm³/mol to about 200, or to about 300, or to about 400 cm³/mol.

Even furthermore, the inventors believe that the stability of a personal-care composition can be further enhanced if one chooses to use personal-care compositions with higher zero-shear viscosities and to avoid personal-care compositions with lower zero-shear viscosities. Preferably, the zero-shear viscosity is at least 500 Pascal-s, or at least 1000 Pascal-s, or at least 1500 Pascal-s.

To enhance the benefit of the present invention, it is important that the partitioned benefit agents incorporated remain stable and do not migrate from one phase to the other. The Partition Coefficient Values (cLogP) reflect a molecule's hydrophilicity and thus the cLogP calculations are considered for the present invention to determine if they are appropriate to resist migration within the particular zones of the present invention. It has been found that partitioned benefit agents with a cLogP greater than 2 will resist migration in personal-care compositions.

cLogP and molar volume can be calculated for a variety of partitioned components with relatively good agreement between the protocols used to calculate them. According to the present invention, the protocol from ACD Labs website was used (www.acdlabs.com). In cases where the partitioned component contains ionizable groups, cLogD (variation of cLogP with pH) is used at the relevant composition pH. ClogP is a calculated quantity for a partitioned component, determined by a mathematical algorithm using molecular substructure or fragment contributions with correction factors. The approach is common in such fields as toxicology, environmental transport, and pharmaceuticals, for example to facilitate development of drugs, especially for topical drugs that interact with lipid bilayers in skin, a molecular mechanism not dissimilar to interaction of partitioned benefit agents with surfactant. Different substructure fragment algorithms exist which can calculate different ClogP values for the same molecule, based on differences in algorithms and/or coefficients, as can be found in scientific literature. For the purposes of our invention, ClogP is determined using the algorithm from Advanced Chemistry Development Labs as referenced and updated in the scientific literature (Hansch, C. and Leo, A., Substituent Constants for Correlation Analysis in Chemistry and Biology, Wiley Interscience New York (1979); updated in Leo., A. and Hoekman, D., Perspect. in Drug Discov. & Design, 18, 19 (2000)), whereas the value of Molar Volume and ClogP were obtained using the ACD/I-lab web service (ACD/Molar Volume 8.02 and ACD/logP 8.02)

Accordingly, the partitioned benefit agents of the present invention may have a cLogP value of at least about 2, at least about 3, at least about 4, or at least about 5. Certain partitioned components, however, are effectively insoluble in either phase thus making it difficult to calculate a cLogP value, which essentially do not migrate, therefore are stable in the zones within the personal-care product. Non-limiting examples of benefit agents along with their cLogP values are charted below accordingly.

TABLE 1
Examples of Partitioned Benefit Agents in the form of
benefit agents with ClogP Values greater than 2.0

| Benefit Agent | cLogP (www.acdlabs.com) |
|---|---|
| Tocopheryl Acetate | 13.63 |
| Isopropyl Myristate | 7.43 |
| Mentol | 3.20 |
| Retinol | 6.84 |
| Isoeugenol | 2.45 |
| Decylene Glycol | 2.38 |
| Titanium Dioxide | >5 |

Additional partitioned benefit materials, which can be used in the personal-care compositions of the present invention, can be selected from the group consisting of preservatives; antimicrobials; fragrances; chelators (e.g. such as those described in U.S. Pat. No. 5,487,884 issued to Bisset, et al.); sequestrants; vitamins (e.g. Retinol); vitamin derivatives (e.g. tocophenyl actetate, niacinamide, panthenol); sunscreens; desquamation actives (e.g. such as those described in U.S. Pat. No. 5,681,852 and 5,652,228 issued to Bisset); anti-wrinkle/anti-atrophy actives (e.g. N-acetyl derivatives, thiols, hydroxyl acids, phenol); anti-oxidants (e.g. ascorbic acid derivatives, tocophenol) skin soothing agents/skin healing agents (e.g. panthenoic acid derivatives, aloe vera, allantoin); skin lightening agents (e.g. kojic acid, arbutin, ascorbic acid derivatives) skin tanning agents (e.g. dihydroxyacteone); essential oils; moisturizing agents (e.g. ); sensates (e.g. menthol); colorants; lather producers (e.g. sodium lauryl sulfate); pH regulators (e.g. triethanolamine) and anti-acne medicaments.

The first personal-care composition may comprise a first concentration of a suspended benefit agent and the second personal-care composition may comprise a second concentration of suspended benefit agent. The suspended benefit agents are selected from the group consisting of comprise hydrophobic benefit materials, polymers, moisturizing agents, pigments, interference pigments, pearlescent agents, particles, exfoliating particles, shiny particles, beads, hydrophobically modified non-platelet particles, microcapsules, and mixtures thereof. The first concentration of suspended benefit agent is different from the second concentration of suspended benefit agent. In another embodiment, the suspended benefit agent in the first personal-care composition is different from the suspended benefit agent in the second personal-care composition. In another embodiment, the second personal-care composition could also comprise a second suspended benefit agent that is different from the suspended benefit agent comprised in the first composition.

Additional suspended benefit agents, which can be used in the personal-care compositions of the present invention, can be selected from the group consisting of microcapsules; thickening agents; low density microspheres (e.g. Expancel 091 WE40 d24, Akzo Nobel and others described in commonly owned and assigned U.S. Patent Publication No. 2004/0092415A1 published on May 13, 2004); polymeric phase structurant (e.g. naturally derived polymers, synthetic polymers, crosslinked polymers, block copolymers, copolymers, hydrophilic polymers, nonionic polymers, anionic polymers, hydrophobic polymers, hydrophobically modified polymers, associative polymers, and oligomers); a liquid crystalline phase inducing structurant (e.g. trihydroxystearin available from Rheox, Inc. under the trade name THIXCIN® R); organic cationic deposition polymer (e.g. Polyquaternium 10 available from Amerchol Corp. Edison, N.J., USA, guar hydroxypropyltrimonium chloride available as Jaguar C-17 from Rhodia Inc., and N-Hance polymer series commercially available from Aqualon); pigments; colorants; pearlescent agents; interference pigments (e.g such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g. talc, kolin, mica, smectite clay, cellulose powder, polysiloxane, silicas, carbonates, titanium dioxide, polyethylene beads) hydrophobically modified non-platelet particles (e.g. hydrophobically modified titanium dioxide and other materials described in a commonly owned, patent application published on Aug. 17, 2006 under Publication No. 2006/0182699A by Taylor, et al.) and mixtures thereof.

The non-limiting list of partitioned benefit agents and suspended benefit agents, illustrated herein are suitable for use in personal-care compositions, and may be incorporated in certain embodiments, for example to assist or enhance cleansing performance, for treatment of the skin, or to modify the aesthetics of the personal-care composition. These components useful in the products herein are described by their cosmetic and/or therapeutic benefit or their postulated mode of action or function. These descriptions are non-limiting and made for the sake of convenience because it is understood that these materials can provide more than one benefit, function or operate via more than one mode of action. The precise nature of these components, and levels of incorporation thereof, will depend on the physical form of the composition and the nature of the cleansing operation for which it is to be used. The amount of partitioned benefit agents or suspended benefit agents in compositions are usually formulated, by weight of the composition, at less than about 10%, less than about 9%, less than about 8%, less than about 7%, less than about 6%, less than about 5%, less than about 4%, less than about 3%, less than about 2%, less than about 1%, less than about 0.5%, less than about 0.25%, less than about 0.1%, less than about 0.01%, less than about 0.005%. Each personal-care composition may comprise from 0.001% to about 0.25%, from about 0.1% to about 0.5%, from about 0.3% from about 1.0%, from about 1.0% to about 10%, from about 2.0% to about 8.0%, from about 3% to about 9.0%, from about 2% to about 5%, by weight of the personal-care composition, of a partitioned benefit agents or suspended benefit agents. In one aspect of the personal-care article of the present invention, the first personal-care composition or the second composition of the present invention may comprise a concentration of 0% partitioned benefit agents or suspended benefit agents.

The compositions of the present invention can be multi-phase and comprise one of more phases or one or more of the components described in the phases below:

The personal-care composition of the present invention can comprise a cleansing phase of components of a cleansing phase. The personal-care composition typically comprises from about 1% to about 100%, by weight of the composition; from about 5% to about 85%; by weight of the composition, from about 10% to 80%, by weight of the composition; from about 20 to 70%, by weight of the composition; from about 25% to 60%, by weight of the composition, from about 30% to about 50%, by weight of the composition, of a cleansing phase.

The cleansing phase can comprise a structured domain that is comprised of a mixture of surfactants. The presence of structured domain enables the incorporation of high levels of hydrophobic benefit materials in a separate phase which is not emulsified within composition. In one aspect, the structured domain in the composition can be characterized as, or is, an opaque structured domain. In one aspect, the opaque structured domain can be characterized as or is, a lamellar phase. The lamellar phase produces a lamellar gel network. The lamellar phase can provide resistance to shear, adequate yield to suspend particles and droplets and at the same time provides long term stability, since it is thermodynamically stable. The lamellar phase tends to have a higher viscosity thus minimizing the need for viscosity modifiers.

In one aspect, cleansing phase can comprise a domain that is comprised of a mixture of surfactants and can be a micellar phase. A micellar phase is optically isotropic. Micelles are approximately spherical in shape. Other shapes such as ellipsoids, cylinders, and bilayers are also possible. In one aspect, the micellar phase can be structured to enhance viscosity and to suspend particles. This can be accomplished using viscosity modifiers such as those defined below as water structurants.

The cleansing phase comprises a surfactant component which can be comprised of a mixture of surfactants including lathering surfactants or a mixture of lathering surfactants. The cleansing phase comprises surfactants suitable for application to the mammalian skin or hair and are compatible with water and the other ingredients of the composition of the present invention. These surfactants include anionic, nonionic, cationic, zwitterionic, amphoteric, soap, or combinations thereof. Preferably, anionic surfactant comprises at least 40% of the surfactant component. The personal-care composition can comprise the surfactant component at concentrations ranging from about 2% to about 40%, from about 4% to about 25%, about 1% to about 21%, about 3 to 15%, by weight of the composition, of the surfactant component.

Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975.

Preferred linear anionic surfactants for use in the structured surfactant phase of the personal-care composition include ammonium lauryl sulfate, ammonium laureth sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof.

Branched anionic surfactants and monomethyl branched anionic surfactants suitable for the present invention are described in a commonly owned, patent application published on December, 2006 under U.S. Publication No. 60/680,149 entitled “Structured Multi-phased Personal-Cleansing Compositions Comprising Branched Anionic Surfactants” filed on May 12, 2005 by Smith, et al. Branched anionic surfactants include but are not limited to the following surfactants: sodium trideceth sulfate, sodium tridecyl sulfate, sodium C₁₂₋₁₃ alkyl sulfate, and C₁₂₋₁₃ pareth sulfate and sodium C₁₂₋₁₃ pareth-n sulfate.

In one aspect of the personal-care compositions of the present invention may further preferably comprise an amphoteric surfactant, a zwitterionic surfactant and mixtures thereof. In one embodiment, the personal-care composition can comprise at least one amphoteric surfactant. Amphoteric surfactant suitable for use in the present invention include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one aspect, the personal-care composition can comprise an amphoteric surfactant that is selected from the group consisting of sodium lauroamphoacetate, sodium cocoamphoactetate, disodium lauroamphoacetate disodium cocodiamphoacetate, and mixtures thereof. Moreover, Amphoacetates and diamphoacetates can also be used.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the personal-care composition include alkyl betaines, including cocoamidopropyl betaine.

The personal-care composition of the present invention is preferably free of alkyl amines and alkanolamide to ensure mildness of the composition to the skin.

An electrolyte can be added per se to the personal-care composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium chloride, ammonium chloride, sodium or ammonium sulfate. The electrolyte is preferably added to the structured surfactant phase of the composition in the amount of from about 0.1% to about 6%; from about 1% to about 5%, more preferably from about 2% to about 4%, more preferably from about 3% to about 4%, by weight of the personal-care composition.

The first personal-care composition can comprise a first concentration of surfactant and second personal-care composition can comprise a second concentration of surfactant. The first concentration of surfactant can be different from the second concentration of surfactant. In one aspect, the first personal-care composition can a first concentration of surfactant that is a greater that the second concentration of surfactant in the second personal-care compositions. In one aspect, the first personal-care composition can have a lower concentration of surfactant than the second personal-care compositions.

The personal-care compositions of the present invention may comprise a benefit phase or benefit phase components. The benefit phase in the present invention is preferably anhydrous and can be substantially free of water. The benefit phase can be substantially free or free of surfactant.

The benefit phase typically comprises hydrophobic benefit materials. The benefit phase may comprise from about 1% to about 50%, preferably from about 5% to about 30%, more preferably from about 10% to about 30%, by weight of the personal-care composition, of a hydrophobic benefit material.

Hydrophobic benefit materials suitable for use in the present invention preferably have a Vaughan Solubility Parameter of from about 5 (cal/cm³)^{1/2} to about 15 (cal/cm³)^{1/2}, as defined by Vaughan in Cosmetics and Toiletries, Vol. 103. The Vaughan Solubility Parameter (VSP) as used herein is a parameter used to define the solubility of hydrophobic materials. Vaughan Solubility parameters are well known in the various chemical and formulation arts and typically have a range of from 5 to 25. Non-limiting examples of hydrophobic benefit materials having VSP values ranging from about 5 to about 15 include the following: Cyclomethicone 5.92, Squalene 6.03, Petrolatum 7.33, Isopropyl Palmitate 7.78, Isopropyl Myristate 8.02, Castor Oil 8.90, Cholesterol 9.55, as reported in Solubility, Effects in Product, Package, Penetration and Preservation, C. D. Vaughan, Cosmetics and Toiletries, Vol. 103, October 1988.

The hydrophobic benefit materials for use in the benefit phase of the composition have a preferred rheology profile as defined by Consistency value (k) and Shear Index (n). The term “Consistency value” or “k” as used herein is a measure of lipid viscosity and is used in combination with Shear Index, to define viscosity for materials whose viscosity is a function of shear. The measurements are made at 35° C. and the units are poise (equal to 100 cps). The term “Shear Index” or “n” as used herein is a measure of lipid viscosity and is used in combination with Consistency value, to define viscosity for materials whose viscosity is a function of shear. The measurements are made at 35° C. and the units are dimensionless. Consistency value (k) and Shear Index (n) are more fully described in the Test Methods below. Preferred Consistency value ranges are 1-10,000 poise (1/sec)ⁿ⁻¹, preferably 10-2000 poise (1/sec)ⁿ⁻¹ and more preferably 50-1000 poise (1/sec)ⁿ⁻¹. Shear Index ranges are 0.1-0.8, preferably 0.1-0.5 and more preferably 0.20-0.4. These preferred rheological properties are especially useful in providing the personal-cleansing compositions with improved deposition of benefit agents on skin.

The benefit phase can be comprised of the hydrophobic benefit materials selected from the group consisting of petrolatum, lanolin, derivatives of lanolin (e.g. lanolin oil, isopropyl lanolate, acetylated lanolin, acetylated lanolin alcohols, lanolin alcohol linoleate, lanolin alcohol riconoleate) hydrocarbon oils (e.g. mineral oil) natural and synthetic waxes (e.g. micro-crystalline waxes, paraffins, ozokerite, lanolin wax, lanolin alcohols, lanolin fatty acids, polyethylene, polybutene, polydecene, pentahydrosqualene) volatile or non-volatile organosiloxanes and their derivatives (e.g. dimethicones, cyclomethicones, alkyl siloxanes, polymethylsiloxanes, methylphenylpolysiloxanes), natural and synthetic triglycerides (e.g. castor oil, soy bean oil, sunflower seed oil, maleated soy bean oil, safflower oil, cotton seed oil, corn oil, walnut oil, peanut oil, olive oil, cod liver oil, almond oil, avocado oil, palm oil, sesame oil) and combinations thereof. In one aspect, at least about 50% by weight of the hydrophobic benefit materials are selected from the groups of petrolatum, mineral oil, paraffins, polyethylene, polybutene, polydecene, dimethicones, alkyl siloxanes, cyclomethicones, lanolin, lanolin oil, lanolin wax. The remainder of the hydrophobic benefit material can be selected from: isopropyl palmitate, cetyl riconoleate, octyl isononanoate, octyl palmitate, isocetyl stearate, hydroxylated milk glyceride and combinations thereof. The benefit phase of the personal-care composition can be comprised a combination of petrolatum and mineral oil.

The personal-care compositions of the present invention can comprise a structured aqueous phase which can comprise a water structurant and water. The structured aqueous phase can be hydrophilic. In one aspect, the structured aqueous phase can be a hydrophilic, non-lathering gelled water phase. The structured aqueous phase can comprises less than about 5%; less than about 3%; less than about 1%, by weight of the structured aqueous phase, of a surfactant component. In one apect, the structured aqueous phase can be is free of lathering surfactants in the composition. The structured aqueous phase of the present invention can comprise from about 30% to about 99%, more than about 50%, more than about 60%, more than about 70%, more than about 80%, by weight of the structured aqueous phase, of water.

The structured aqueous phase which can comprise in some aspects a water structurant. The water structurant is selected from the group consisting of inorganic water structurants (e.g. silicas, polyacrylates, polyacrylamides, modified starches, crosslinked polymeric gellants, copolymers) charged polymeric water structurants (e.g. Acrylates/Vinyl Isodecanoate Crosspolymer (Stabylen 30 from 3V), Acrylates/C10-30 Alkyl Acrylate Crosspolymer (Pemulen TR1 and TR2), Carbomers, Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), water soluble polymeric structurants (e.g. cellulose gums and gel, and starches), associative water structurants (e.g. xanthum gum, gellum gum, pectins, alginates such as propylene glycol alginate), and mixtures thereof. The structured aqueous phase can comprise from about 0.1% to about 30%, from about 0.5% to about 20%, from about 0.5% to about 10%, and from about 0.5% to about 5%, by weight of the structured aqueous phase, of a water structurant. A water structurant for the structured aqueous phase can have a net cationic charge, net anionic charge, or neutral charge.

The structured aqueous phase can have a pH in the range from about 5 to about 9.5, or in one aspect have a pH of about 7. The structured aqueous phase of the present compositions can further comprise optional ingredients such as, pigments, pH regulators (e.g. triethanolamine), and preservatives.

Other optional ingredients are most typically those materials approved for use in cosmetics and that are described in the CTFA Cosmetic Ingredient Handbook, Second Edition, The Cosmetic, Toiletries, and Fragrance Association, Inc. 1988, 1992.

### Test Methods

### Benefit Analysis Method:

This method determines the weight ratio of cleansing (surfactant) phase to lipid phase in dual phase composition. A sample of dual-phase composition is mixed and tested using a moisture analyzer for % moisture. The result is calculated by dividing the total % moisture in the composition by the % moisture in the surfactant phase then multiplying that result by 100. The % benefit agent (lipid) is calculated by subtracting the % surfactant phase from 100. It is applicable only to dual phase compositions in which one phase (lipd) contributes no volatiles at the temperature conditions used in the instrument program.

Apparatus:

| | |
|---|---|
| Infared or Halogen | Programed according to the operating manual, |
| Moisture Balance (e.g. | using the following test parameters: |
| Mettler-Toledo HR73 | Heating mode: Ramp to 140° C. in 5 minutes |
| Moisture Analyzer) | Switchoff mode: 3 |
| aluminum drying pans | Aluminum drying pans must be dried and |
| (4 inch × 5/16 inch | stored in a desiccator prior to use. Dry the |
| deep) | pans in a conventional oven for 1 hour at |
| | 130° C. Allow pans to cool to room |
| | temperature before using. Always handle pans |
| | with a clean pair of forceps |
| 1 ml Disposable Syringes | |
| Analytical Balance | capable of weighing to 3 decimal places |

### Dialysis Method:

The Dialysis Method is for determining the migration, or diffusion over time, of chemical partitioned components from one composition of a dual-composition system to a second composition of a dual-composition system. It is designed for viscous materials. Migration is accelerated using a cell with two chambers divided by a dialysis cell, as described below. The bulk of the compositions are kept separate but molecules smaller than 3,500 MW are free to diffuse. The high surface area to thickness ratio allows diffusion to go to equilibrium in a manageable time frame. The materials needed are: a dialysis cell (described below), a dialysis membrane composed of regenerated cellulose with a molecular weight cut off of 3,500, available from Pierce Biotechnology of Thermo Fisher Scientific (Pierce Biotechnology, Inc.; P.O. Box 117; Rockford, Ill. 61105 product no. 68035) which is cut open to lay flat; clamps; disposable syringes; and a flat-edged spatula In the case of testing from a product package, two zones can be selected from the package that contains at least two compositions that contain the separate partitioned or suspended benefit agents. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

Loading and Unloading compositions into dialysis cell: A first endplate made of Plexiglas TM having the dimensions of 6 inches in length, 5 inches in width and ½ inch depth is placed on a flat surface and topped with first gasket made of silicone rubber having same dimensions as end plate, with a cutout in the center that has the dimension of 4 inches in length by 1½ inches in width. The gasket is pressed down to form a seal with the endplate, then 20 grams of the first composition in a disposable syringe is dispensed into the space in the gasket. The dialysis membrane, having similar in dimensions to endplate and the first gasket, is placed on top of this and pressed down to form a seal with the first gasket. A second gasket made of the same material and same dimensions as the first gasket is placed on top of the dialysis membrane and pressed down. The second composition is then dispensed into the space in the second gasket on top of the dialysis membrane. This is topped with the second endplate, having dimensions and made similar in materials as the first endplate, and the entire assembly is held together with clamps. It can be placed vertically on a flat surface for the duration of 1 week at 25° C. To remove the test materials, place the diffusion cell flat and disassemble in the reverse order, scraping each material out with a flat-edged spatula as it is exposed. Each composition is analyzed individually for partitioned components according to the Gas Chromatograph Method described hereinafter.

### Gas Chromatograph Method:

### Menthol, Triethyl Citrate and Isopropyl Myristate Analysis Parameters

Solutions:

| | |
|---|---|
| Internal Standard | Dissolved 300 mg diphenyl oxide in 200 ml methanol. |
| Solution 1 | |
| (ISTD1): | |
| Standard Stock | Dissolved 100 mg each of menthol, triethyl citrate |
| Solution: | and isopropyl myristate in 100 mL methanol. |
| Calibration | Combined 10.0 mL standard stock solution with |
| Solution: | 10.0 mL ISTD1 and 80 mL methanol. |

Sample Preparation: Weigh 4 g of the composition and disperse in 90 mL of methanol, with sonication and gentle warming. 10.0 mL is added of the ISTD1. This combination is filtered through Acrodisc syringe filter (PVDF, 25 mm diam, 0.45 um pore size). If unable to filter through the PVDF, 25 mm diam. 0.45 um pore size filter, sample solutions may be prefiltered through Glass Fiber Acrodisc syringe filter (37 mm, 1 um pore size).

Instrument Parameters:

Gas Chromatograph:

| | |
|---|---|
| Column | DB-5HT, 15 M × 0.32 mm I.D., 0.10 um film |
| | thickness (J&W cat. no. 123-5711) |
| Column Flow | 2.5 ml hydrogen/min, constant flow mode |
| | (velocity 54 cm/sec) |
| Injector Temp | 250° C. |
| Injection Mode | 1 ul, split @ 25:1 |
| Oven Program | Initial temp 80° C., ramp 30°/min to 200°, |
| | then 15°/min to 315°, final temp 315°, |
| | final time 2.0 min. |
| FID Temp | 325° |
| Detector Flows | Hydrogen: 40 mL/min |
| | Air: 400 mL/min |
| | Mode: constant col + makeup flow |
| | Combined flow: 35 mL/min |
| | Makeup gas: nitrogen |

### Glycerin and Caprylyl Glycol Analysis Parameters

Solutions:

| | |
|---|---|
| Internal Standard | Tridecanol 10 mg/mL methanol |
| Solution 2 | |
| (ISTD2): | |
| Standard Stock | Dissolved 100 mg each of glycerin and caprylyl |
| Solution: | glycol in 100 mL methanol. |
| Calibration | Combined 10.0 mL standard stock solution with |
| Solution: | 1.0 mL ISTD2 and 90 mL methanol. Derivatized |
| | with Sylon BFT as in method. |

Sample Preparation: Weigh 4 g of the composition and disperse in 90 mL of methanol, with sonication and gentle warming. 1.0 mL of ISTD2 is added. The combination is derivatized with Sylon BFT, as in method.

Instrument Parameters:

Gas Chromatograph

| | |
|---|---|
| Column | DB-5HT, 15 M × 0.32 mm I.D., 0.10 um film |
| | thickness (J&W cat. no. 123-5711) |
| Column FLow | 2.5 ml hydrogen/min, constant flow mode |
| | (velocity 54 cm/sec) |
| Injector Temp | 250° C. |
| Injection Mode | 1 ul, split @ 25:1 |
| Oven Program | Initial temp 80° C., ramp 30°/min to 200°, |
| | then 15°/min to 315°, final temp 315°, |
| | final time 2.0 min. |
| FID Temp | 325° |
| Detector Flows | Hydrogen: 40 mL/min |
| | Air: 400 mL/min |
| | Mode: constant col + makeup flow |
| | Combined flow: 35 mL/min |
| | Makeup gas: nitrogen |

### Vitamin E Acetate Analysis Parameters

| | |
|---|---|
| Mobile Phase: | Mixed 300 mL 2-propanol with 700 mL methanol. |
| Internal Standard | Dissolved 125 mg Vitamin K1 in 250 ml mobile |
| Solution 3 | phase. |
| (ISTD3): | |
| Stock Standard | Dissolved 25 mg Vitamin E Acetate in 25 mL |
| Solution: | mobile phase. |
| Calibration | Mixed 1.0 mL Vitamin E Acetate stock solution |
| Solution: | with 2.0 mL ISTD3 and 7 mL mobile phase. |

Sample Preparation: Weigh 200 mg of the composition and disperse into 8 mL of the mobile phase and 2.0 mL of ISTD3 is added. The mixture is filtered through a Whatman GDX 0.45 um filter for HPLC analysis.

Instrument Parameters:

| | |
|---|---|
| HPLC System | HPLC pump, liquid autosampler, UV detector and a |
| | computing integrator or a PC-based data system. |
| Column | Alltima C18 Peek, 5 um, 250 mm × 4.6 mm (Alltech |
| | cat. no. 88055) |
| Column Temp | approx. 25° C. (ambient) |
| UV Wavelength | 280 nm |
| Injection Volume | 20 uL |
| Flow Rate | 1.0 mL/min |
| Run Time | 22.0 minutes |
| Retention Times | Vitamin E Acetate 11.2 min |
| | Vitamin K1 15.1 min |

Operation of the Gas Chromatograph: The calibration solution is injected. The peaks of interest are identified and the instrument is calibrated. The sample solutions are injected sample solutions and calibrated peaks are quantified.

### T-Bar Viscosity Method:

The viscosity of a composition contained in a zone can be assessed in by the T-Bar Viscosity Method. In the case of testing from a product package, two zones can be selected from the package that contains at least two compositions that contain the separate partitioned or suspended benefit agents. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions. The apparatus for T-Bar measurement includes a Brookfield DV-II+ Pro Viscometer with Helipath Accessory; chuck, weight and closer assembly for T-bar attachment; a T-bar Spindle D, a personal-computer with Rheocalc software from Brookfield, and a cable connecting the Brookfield Viscometer to the computer. First, weigh 80 grams of the first or second composition in a 4-oz glass jar. Measure the T-bar viscosity by carefully dropping the T-Bar Spindle to the interior bottom of the jar and set the Helipath stand to travel in an upward direction. Open the Rheocalc software and set the following data acquisition parameters: set Speed to 5 rpm, set Time Wait for Torque to 00:01 (1 second), set Loop Start Count at 100. Start data acquisition and turn on the Helipath stand to travel upward at a speed of 22 mm/min. The T-Bar viscosity “T,” is the average T-Bar viscosity reading between the 6^{th} reading and the 95^{th} reading (the first five and the last five readings are not used for the average T-Bar viscosity calculation). If the viscosity is below the lower limit of the D spindle (30,000 cps), a larger spindle can be used for the T-Bar Viscosity measurement.

### Ultracentrifugation Method:

The Ultracentrifugation Method is used to determine the percent of a structured domain or an opaque structured domain that is present in a multi-phase personal-care composition that comprises a structured surfactant phase comprising a surfactant component. The method involves the separation of the composition by ultracentrifugation into separate but distinguishable layers. The multi-phase personal-care composition of the present invention can have multiple distinguishable layers, for example a non-structured surfactant layer, a structured surfactant layer, and a benefit layer.

First, dispense about 4 grams of multi-phase personal-care composition into Beckman Centrifuge Tube (11×60 mm). Next, place the centrifuge tubes in an Ultracentrifuge (Beckman Model L8-M or equivalent) and ultracentrifuge using the following conditions: 50,000 rpm, 18 hours, and 25° C.

After ultracentrifuging for 18 hours, determine the relative phase volume by measuring the height of each layer visually using an Electronic Digital Caliper (within 0.01 mm). First, the total height is measured as Hₐ which includes all materials in the ultracentrifuge tube. Second, the height of the benefit layer is measured as H_{b}. Third, the structured surfactant layer is measured as H_{c}. The benefit layer is determined by its low moisture content (less than 10% water as measured by Karl Fischer Titration). It generally presents at the top of the centrifuge tube. The total surfactant layer height (Hₛ) can be calculated by this equation:

Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic phase generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. Structured surfactant layer display multiple lines that are often associated primarily with the long spacings of the liquid crystal structure. There may be several structured layers present, so that H_{c} is the sum of the individual structured layers. If a coacervate phase or any type of polymer-surfactant phase is present, it is considered a structured phase.

Finally, the structured domain volume ratio is calculated as follows:

Structured Domain Volume Ratio=_{c}/Hₛ*100%

If there is no benefit phase present, use the total height as the surfactant layer height, Hₛ=Hₐ.

### Yield Stress and Zero Shear Viscosity Method:

The Yield Stress and Zero Shear viscosity of a composition contained in a zone can be assessed by the Yield Stress and Zero Shear Viscosity method. In the case of testing from a product package, two zones can be selected from the package that contains at least two compositions that contain the separate partitioned or suspended benefit agents. In order to separate the zones, the product can be frozen at a temperature of at least −20° C. for a period of at least 24 hours. The zones are then cut using a cutting implement such as a bandsaw. The cut portions are collected separately and allowed equilibrate to ambient conditions.

A controlled stress rheometer such as a TA Instruments AR2000 Rheometer is used to determine the Yield Stress and Zero Shear Viscosity. The determination is performed at 25° C. with the 4 cm diameter parallel plate measuring system and a 1 mm gap. The geometry has a shear stress factor of 79580 m⁻³ to convert torque obtained to stress. Serrated plates can be used to obtain consistent results when slip occurs.

First a sample of the composition is obtained and placed in position on the rheometer base plate, the measurement geometry (upper plate) moving into position 1 mm above the base plate. Excess composition at the geometry edge is removed by scraping after locking the geometry. If the composition comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a composition has any particle larger than 5 mm in any dimension, the particles are removed prior to the measurement.

The determination is performed via the programmed application of a continuous shear stress ramp from 0.1 Pa to 1,000 Pa over a time interval of 4 minutes using a logarithmic progression, i.e., measurement points evenly spaced on a logarithmic scale. Thirty (30) measurement points per decade of stress increase are obtained. Stress, strain and viscosity are recorded. If the measurement result is incomplete, for example if material flows from the gap, results obtained are evaluated and incomplete data points excluded. The Yield Stress is determined as follows. Stress (Pa) and strain (unitless) data are transformed by taking their logarithms (base 10). Log(stress) is graphed vs. log(strain) for only the data obtained between a stress of 0.2 Pa and 2.0 Pa, about 30 points. If the viscosity at a stress of 1 Pa is less than 500 Pa-sec but greater than 75 Pa-sec, then log(stress) is graphed vs. log(strain) for only the data between 0.2 Pa and 1.0 Pa, and the following mathematical procedure is followed. If the viscosity at a stress of 1 Pa is less than 75 Pa-sec, the zero shear viscosity is the median of the 4 highest viscosity values (i.e., individual points) obtained in the test, the yield stress is zero, and the following mathematical procedure is not used. The mathematical procedure is as follows. A straight line least squares regression is performed on the results using the logarithmically transformed data in the indicated stress region, an equation being obtained of the form:

Log(strain)=m*Log(stress)+b (1)

Using the regression obtained, for each stress value (i.e., individual point) in the determination between 0.1 and 1,000 Pa, a predicted value of log(strain) is obtained using the coefficients m and b obtained, and the actual stress, using Equation (1). From the predicted log(strain), a predicted strain at each stress is obtained by taking the antilog (i.e., 10^{x} for each x). The predicted strain is compared to the actual strain at each measurement point to obtain a % variation at each point, using Equation (2).

% variation=100*(measured strain−predicted strain)/measured strain (2)

The Yield Stress is the first stress (Pa) at which % variation exceeds 10% and subsequent (higher) stresses result in even greater variation than 10% due to the onset of flow or deformation of the structure. The Zero Shear Viscosity is obtained by taking a first median value of viscosity in Pascal-seconds (Pa-sec) for viscosity data obtained between and including 0.1 Pa and the Yield Stress. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

### Examples

### Example 1

Example 1 including composition A and Composition C containing six benefit agents and Composition B and Composition D not containing six benefit agents. Composition A and Composition B were made by conventional mixing techniques in the order of addition indicated. Addition step 8 in Table 2 containing Tridecyl Alcohol, PEG-90M, Xanthan Gum, and Hydroxypropyl Guar and addition step 11 in Table 2 containing Tocopheryl Acetate, Isopropyl Myristate, Menthol, Triethyl Citrate, Caprylyl Glycol, and Glycerine was premixed prior to addition to the batch.

TABLE 2

| | | (%) of Ingredients | |
|---|---|---|---|
| | | in Composition | |
| Composition Ingredients | | Composition A | Composition B |
| 1. | Water | QS | QS |
| 2. | Sodium Trideceth-3 Sufate | 8.5 | 8.5 |
| 3. | Sodium Lauryl Sulfate | 8.5 | 8.5 |
| 4. | Sodium Lauroamphoacetate | 5.0 | 5.0 |
| 5. | Disodium EDTA | 0.15 | 0.15 |
| 6. | Sodium Benzoate | 0.2 | 0.2 |
| 7. | Sodium Chloride | 4.75 | 4.75 |
| 8. | PEG-90M | 0.15 | 0.15 |
| | Xanthan Gum | 0.22 | 0.22 |
| | Tridecyl Alcohol | 2 | 2 |
| | Hydroxypropyl Guar | 0.6 | 0.6 |
| 9. | Citric Acid | 1 | 1 |
| 10. | Methyl Chloro Isothiazolinone & | 0.0005 | 0.0005 |
| | Methyl Isothiazolinone | | |
| 11. | Tocopheryl Acetate | 0.5 | — |
| | Isopropyl Myristate | 0.5 | — |
| | Menthol | 0.5 | — |
| | Triethyl Citrate | 0.5 | — |
| | Caprylyl Glycol | 0.5 | — |
| | Glycerine | 0.5 | — |

Composition C and Composition D were made by conventional mixing techniques in the order of addition indicated. Addition step 6 in Table 3 containing Water and Polyquaternium-10 and addition step 11 in Table 3 containing Tocopheryl Acetate, Isopropyl Myristate, Menthol, Triethyl Citrate, Caprylyl Glycol, and Glycerine are premixed prior to addition to the main batch.

TABLE 3
Composition C and D

| | | (%) of Ingredients | |
|---|---|---|---|
| | | in Composition | |
| Composition Ingredients | | Composition C | Composition D |
| 1. | Sodium Lauryl Sulfate | 9.5 | 9.5 |
| 2. | Sodium Laureth Sulfate | 5.7 | 5.7 |
| | Adjust to pH 5 with Citric Acid | | |
| 3. | Acrylates Copolymer (Aqua SF-1) | 1.9 | 1.9 |
| 4. | Coco Monoethanolamide | 0.76 | 0.76 |
| 5. | Caustic Soda 50% | 0.16 | 0.16 |
| 6. | Water | QS | QS |
| | Polyquaternium-10 | 0.24 | 0.24 |
| 7. | Disodium EDTA | 0.12 | 0.12 |
| 8. | Sodium Benzoate | 0.24 | 0.24 |
| 9. | Citric Acid | 0.65 | 0.65 |
| 10. | Methyl Chloro Isothiazolinone & | 0.0005 | 0.0005 |
| | Methyl Isothiazolinone | | |
| 11. | Tocopheryl Acetate | 0.5 | — |
| | Isopropyl Myristate | 0.5 | — |
| | Menthol | 0.5 | — |
| | Triethyl Citrate | 0.5 | — |
| | Caprylyl Glycol | 0.5 | — |
| | Glycerine | 0.5 | — |

After the compositions were made, Composition A and Composition B were placed in a one dialysis cell according to the dialysis method and Composition C and Composition D were placed in a dialysis cell according to the dialysis method. Compositions A and B had a zero-shear viscosity of 5882 and compositions C and D had a zero-shear viscosity of 543 Pa·s

Compositions B and D were analyzed according to the Gas Chromatograph method. The migration of the benefit agents, Tocopheryl Acetate, Isopropyl Myristate, Menthol, Triethyl Citrate, Caprylyl Glycol, and Glycerine were analyzed in each of the compositions. Composition B and D were analyzed for the benefit agents that had migrated from Compositions A and C. The results of the Gas Chromatograph are shown in Table 4 below. Results showed that benefit agents with low ClogP components have a greater tendency to migrate than benefit agents with a high ClogP. Furthermore, results indicate that benefit agents contained in a composition with a higher zero-shear viscosity migrate has less migration than benefit agents contained in a composition with lower zero-shear viscosity.

TABLE 4
Percent migration of benefit agents from Composition
A and C as analyzed in Composition B and D

| Molar | | | Percent Migration | Percent Migration |
|---|---|---|---|---|
| Volume | CLogP | Benefit Agent | from A to B | from C to D |
| 498 | 13.63 | Tocopheryl Acetate | Not Detected | Not Detected |
| 313 | 7.43 | Isopropyl Myristate | 2.4 | Not Detected |
| 176 | 3.20 | Menthol | 9.8 | 16.7 |
| 235 | 1.49 | Triethl Citrate | Equilibrium | 42.6 |
| | | | (>50%) | |
| 139 | 0.78 | Caprylyl Glycol | 22.7 | 31.4 |
| 71 | −2.32 | Glycerine | Equilibrium | Equilibrium |
| | | | (>50%) | (>50%) |

The inventor was able to conclude from this data that benefit agents Tocopheryl Acetate, Isopropyl Myristate, and Menthol could be considered stable partitioned benefit agents. Caprylyl Glycol could also be considered a stable benefit agent in a system with a zero-shear viscosity of 5882 Pa·s. These components are stable and can be used in a two composition system and not be expected to migrate.

### Example 2

Example 2 is a personal-care product containing composition E, which contained blue exfoliating beads, and composition F, which contained red exfoliating beads. Composition E and Composition F were made by conventional mixing techniques in the order of addition indicated. Addition step 6 in Table 5 containing water and polyquaternium-10 is premixed prior to addition to the main batch.

TABLE 5
Composition E and F

| | | (%) of Ingredients | |
|---|---|---|---|
| | | in Composition | |
| Composition Ingredients | | Composition E | Composition F |
| 1. | Sodium Lauryl Sulfate | 9.5 | 9.5 |
| 2. | Sodium Laureth Sulfate | 5.7 | 5.7 |
| | Adjust to pH 5 with Citric Acid | | |
| 3. | Acrylates Copolymer | 1.9 | 1.9 |
| | (Aqua SF-1) | | |
| 4. | Coco Monoethanolamide | 0.76 | 0.76 |
| 5. | Caustic Soda 50% | 0.16 | 0.16 |
| 6. | Water | QS | QS |
| | Polyquaternium-10 | 0.24 | 0.24 |
| 7. | Disodium EDTA | 0.12 | 0.12 |
| 8. | Sodium Benzoate | 0.24 | 0.24 |
| 9. | Citric Acid | 0.65 | 0.65 |
| 10. | Methyl Chloro Isothiazolinone & | 0.0005 | 0.0005 |
| | Methyl Isothiazolinone | | |
| 11. | Fragrance | 1 | 1 |
| 12. | Oxidized Polyethylene - Blue | 0.3 | — |
| | Beads ¹ | | |
| 13. | Oxidized Polyethylene - Red | — | 0.3 |
| | Beads ² | | |

¹ Oxidized Polyethylene BU305 - Blue Beads: Supplied by Accutech, LLC; 325 Spring Street; Clinton, TN 37716; Density = 0.98 g/cm³
² Oxidized Polyethylene - Red Beads: Supplied by Baker and Hughes; 9100 W. 21st Street; Sand Springs, Oklahoma 74063; Density = 0.98 g/cm³

100 ml of Composition E was filled in an 8 oz. glass jar. 100 ml of composition F were then layered on top of composition E in the same 8 oz. glass jar. The product had a visual appearance which had a zone containing blue beads on the bottom and a zone containing red beads on the top. The 8 oz. glass jar was placed in 120° F. for a period of 10 days. After 10 days at 120° F., there was no mixing of the red beads with the blue beads and they remained stable in their respective zones.

From this work, the inventors were able to conclude that larger molecules or particles could remain separated in a product that contained two separate compositions.

Although the preceding description and examples are generally tailored to liquid personal-care products, a person skilled in the art would know how to make other personal-care products-e.g. solid, semi-solid, semi-liquid, cream, lotion, gel-comprising at least two compositions each having a concentration of a partitioned benefit agent or suspended benefit agent which is noticeably distinct from the other.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A personal-care article for dispensing and or applying a personal-care product comprising
a) a package comprising at least one chamber, a dispensing orifice, a first zone proximate to said dispensing orifice, and a second zone distal to said dispensing orifice, wherein said first zone and said second zone are both located in said at least one chamber; and
b) a personal-care product comprising a first personal-care composition substantially disposed within said first zone and a second personal-care composition substantially disposed within said second zone;
wherein said first composition comprises a first concentration of a partitioned benefit agent;
wherein said second composition comprises a second concentration of said partitioned benefit agent;
wherein said partitioned benefit agent is selected from the group consisting of fragrances, moisturizing agents, lather producers, vitamins, vitamin derivatives, sunscreens, anti-wrinkle, skin soothing agents, skin lightening agents, skin tanning agents, anti-acne medicaments, essential oils, sensates, feel agents, colorants, and mixtures thereof; and
wherein said first concentration is different from said second concentration.

2) The personal-care article of claim 1, wherein said first concentration of said partitioned benefit agent is greater than said second concentration of said partitioned benefit agent.

3) The personal-care article of claim 1, wherein said second concentration of said partitioned benefit agent is greater than said first concentration said partitioned benefit agent.

4) The personal-care article of claim 1, wherein said first zone comprises from about 10% to about 90%, by volume, of said package.

5) The personal-care article of claim 1, wherein said first zone comprises from about 30% to about 70%, by volume of said package.

6) The personal-care article of claim 1, wherein said first zone comprises about 50%, by volume of said package.

7) The personal-care article of claim 1, wherein said first zone is in physical contact with said second zone with said package.

8) The personal-care article of claim 1, wherein said first personal-care composition comprises a first concentration of surfactant and said second personal-care composition comprises a second concentration of surfactant.

9) The personal-care article of claim 8, wherein said first concentration of surfactant is greater than said second concentration of surfactant.

10) The personal-care article of claim 1, wherein said first personal-care composition comprises a suspended benefit agent selected from the group consisting of hydrophobic benefit materials, polymers, moisturizing agents, pigments, interference pigments, pearlescent agents, particles, exfoliating particles, shiny particles, beads, hydrophobically modified non-platelet particles, microcapsules, and mixtures thereof.

11) The personal-care article of claim 1, wherein said second personal-care composition comprises a suspended benefit agent selected from the group consisting of hydrophobic benefit materials, polymers, moisturizing agents, pigments, interference pigments, pearlescent agents, particles, exfoliating particles, shiny particles, beads, hydrophobically modified non-platelet particles, microcapsules, and mixtures thereof.

12) The personal-care article of claim 1, wherein said first personal-care composition comprises a lamellar phase.

13) The personal-care article of claim 1, wherein said second personal-care composition comprises a lamellar phase.

14) The personal-care article of claim 1, wherein said package further comprises a third zone medial to said dispensing orifice.

15) The personal-care article of claim 14, wherein said product further comprises a third personal-care composition substantially disposed within said third zone; said third personal-care composition comprising a third concentration of a partitioned benefit agent wherein said third concentration is different from said first concentration and said second concentration.

16) A personal-care article for dispensing and or applying a personal-care product comprising:
a) a package comprising at least one chamber, a dispensing orifice, a first zone proximate to said dispensing orifice, and a second zone distal to said dispensing orifice, wherein said first zone and said second zone are both located in said at least one chamber; and
b) a personal-care product comprising a first personal-care composition substantially disposed within said first zone and a second personal-care composition substantially disposed within said second zone;
wherein said first composition comprises a first concentration of a suspended benefit agent;
wherein said second composition comprises a second concentration of said suspended benefit agent;
wherein said suspended benefit agent is selected from the group consisting of hydrophobic benefit materials, polymers, moisturizing agents, pigments, interference pigments, pearlescent agents, particles, exfoliating particles, shiny particles, beads, hydrophobically modified non-platelet particles, microcapsules, and mixtures thereof; and
wherein said first concentration is different from said second concentration.

17) The personal-care article of claim 16, wherein said first concentration of said suspended benefit agent is greater than said second concentration of said suspended benefit agent.

18) The personal-care article of claim 16, wherein said second concentration of said suspended benefit agent is greater than said first concentration said suspended benefit agent.

19) The personal-care article of claim 16, wherein said first personal-care composition further comprises a partitioned benefit agent selected from the group consisting of fragrances, moisturizing agents, lather producers, vitamins, vitamin derivatives, sunscreens, anti-wrinkle, skin soothing agents, skin lightening agents, skin tanning agents, anti-acne medicaments, essential oils, sensates, colorants, and mixtures thereof.

20) The personal-care article of claim 16, wherein said second personal-care composition further comprises a partitioned benefit agent selected from the group consisting of fragrances, moisturizing agents, lather producers, vitamins, vitamin derivatives, sunscreens, anti-wrinkle, skin soothing agents, skin lightening agents, skin tanning agents, anti-acne medicaments, essential oils, sensates, feel agents, colorants, and mixtures thereof.
